(19) United States
(12) Patent Application Publication
(10) Pub. No.: US 2006/0058638 A1
(43) Pub. Date: Mar. 16, 2006

(54) Method and device for the diagnosis and treatment of aortic aneurysms

(75) Inventors: Jan Boese, Eckental (DE); Martin Kleen, Furth (DE); Klaus Klingenbeck-Regn, Numberg (DE); Michael Maschke, Lonnerstadt (DE)

Correspondence Address: SIEMENS CORPORATION;INTELLECTUAL PROPERTY DEPARTMENT, 170 WOOD AVENUE, SOUTH, ISELIN, NJ, 08830, US

(73) Assignee: Siemens Aktiengesellschaft

(21) Appl. No.: 11/222,394

(22) Filed: Sep. 8, 2005

(30) Foreign Application Priority Data
Sep. 14, 2004 (DE) 10 2004 044 435.8

(51) Int. Cl. A61B 5/05

(52) U.S. Cl. 600/411

(57) ABSTRACT

Method for the diagnosis and treatment of aortic aneurysms through representation of the affected area of the aorta by means of an imaging method and measurement of the image in order to produce a made-to-measure stent graft which is introduced into the aorta in the folded state and then expanded, a digital model of the aortic aneurysm being created from the images present as 3D volume data, from which digital model of the aortic aneurysm there is produced by means of iterative customizing software a digital model of the stent graft on the basis of which the stent graft is manufactured.

The invention relates to a method for the diagnosis and treatment of aortic aneurysms through representation of the affected area of the aorta by means of an imaging method and measurement of the image for the purpose of fabricating a made-to-measure stent graft which is introduced into the aorta in the folded state and then expanded, and a device for performing said method.

The diagnosis and treatment of aortic aneurysms has increased significantly in importance over the last several years. An aortic aneurysm is a dilatation of the aorta caused by a weakness in the vessel wall. Aortic aneurysm is an atherosclerotic disease. Atherosclerosis is considered the main cause of cardiovascular diseases, which in turn are the most common cause of death in the western world. In den USA, for example, the prevalence of aortic aneurysm is approximately 1.5% in persons over 50 years of age, with an untreated aneurysm carrying a high risk of rupture. For example, the probability of an aortic aneurysm with a diameter greater than 5 cm bursting within a 5-year period is 20%, this being linked in turn to a mortality of approximately 80%.

Even up to a few years ago the diagnosis was performed exclusively by radiologists and the treatment exclusively operatively by surgeons. In more recent times an increasing interest has developed in having diagnosis and treatment carried out in a medical unit by an interdisciplinary medical team. In the optimal case the aim is to perform the treatment, for example the implantation of a stent graft, as minimally invasively as possible, that is to say interventionally using a catheter-based procedure.

According to the current prior art there is no fully integrated solution for diagnosis and treatment, simultaneously or shortly thereafter, of an aortic aneurysm. Either a medical apparatus is suitable for the diagnosis, such as a computer tomograph for example, or else it is suitable for the treatment, such as an angiography system for example.

An apparatus has become known from DE 199 01 482 which permits a patient lying on a movable patient support table to be examined in different imaging systems without changing beds. The German publication Fortschr Röntgenstr 2004: 176:56-61 “Blutflusssimulation mittels Computational-Fluid-Dynamics an aus CT-Daten rekonstruierten Aortenaneurysmata vor und nach Stent-Graft Implantation” (“Blood flow simulation using computational fluid dynamics on aortic aneurysms reconstructed from CT data before and after stent graft implantation”) discusses a solution which performs a blood flow simulation of the abdominal aorta before and after the stent implantation based on multi-detector row datasets. This solution too represents only a partial solution in the area of diagnosis and/or therapy planning.

Previously there has been known, for example, a diagnosis and therapy method wherein the aortic aneurysm is measured directly in the images with the aid of a scale in terms of length, width etc. in order to produce a more or less matching stent graft according to these dimensions and then implant same. Considering that there are located in the area of an aortic aneurysm of said type a plurality of outgoing arteries which must of course be kept free, which is to say that the stent graft must have corresponding cutouts, it is clear that a truly made-to-measure stent graft cannot usefully be manufactured in this way.

The object of the invention is therefore to create a method and a device suitable for performing said method by means of which an integrated treatment of an aortic aneurysm will be possible starting from the diagnosis through to the stent manufacture.

In order to achieve this object, in a method of the type referred to at the beginning it is provided according to the invention that a digital model of the aortic aneurysm is constructed from the images present as 3D volume data, from which digital model of the aortic aneurysm there is produced by means of iterative customizing software a digital model of the stent graft on the basis of which the stent graft is manufactured, the aim here being to manufacture the stent graft by means of an automatic or partially automated system for producing vascular prostheses from digital representations, as is already known in the other context, so that the actual therapeutic treatment can be performed through insertion of the made-to-measure stent graft thus obtained as close as possible in time relative to the diagnosis of the aortic aneurysm.

In a development of the invention it can be provided here that at the time of insertion of the stent graft image data generated during the intervention is fused with the digital model of the stent graft and/or the digital model of the aortic aneurysm in order to enable the actual position and expansion of the stent graft to be compared with the planned position and expansion and so permit an optimal positioning of the stent graft which is introduced for example by way of a catheter.

In this case a CT device, an MR device or an angiography device, as has also already been used for the diagnosis, is used to insert the stent graft.

In order to perform the method according to the invention a device is provided which is characterized by a workstation which is connected to all the imaging systems used as well as to an automatic or partially automated manufacturing station for producing the made-to-measure-stent graft, and which comprises a segmentation program for producing a digital model of the aortic aneurysm and an iterative customizing program for producing a digital model of the stent graft with the aid of the digital model of the aortic aneurysm, as well as a memory device and a display device for visually presenting the images.

The device according to the invention is a system which integrates different imaging subsystems, medical measuring equipment including coordinated user interfaces to form a medical unit in order to allow a complete aortic aneurysm procedure consisting of diagnosis, therapy planning, therapy and progress monitoring. This includes the following subsystems:
X-ray angiography system.
Ultrasound system to support the imaging and possible flow measurement.
CT and/or MR system.
Imaging system for capturing, editing, reconstructing, communicating and archiving 2D, 3D and 4D image data.
Imaging system for fusing medical image data including physiological measurement data (e.g. ECG, pressure or flow, including from optional ventilator and/or anesthesia system). This data can be 2D, 3D and 4D. The fusion imaging system contains solutions for manual and automatic segmentation and registration of medical data. Solutions for this purpose are known to the person skilled in the art.
Modules for ECG and breath gating to suppress artifacts.
Digital memory device and interface to medical data networks (DICOM), HIS, RIS.
2D and 3D display and/or projection devices in order to display the medical data in an optimal manner.
Automatic or partially automated system for producing vascular prostheses (stent grafts) from digital representations.
Documentation system for seamlessly documenting all the steps.

After the recording of diagnostic volume data (e.g. MR, CT, 3D rotation angiography, ultrasound), the following steps can occur in different sequences and combinations during the planning of the intervention:
1. Generation of a digital representation of the aortic aneurysm.
2. Generation of a digital representation of the stent graft for treatment of the aortic aneurysm.
3. Decision on the use of a prefabricated stent graft or a stent to be custom-made.
4. Checking of the dimensional accuracy of the digital representation of the stent graft (e.g. by overlaying the digital representation of the stent graft with the volume data). Possible modification of the digital representation of the stent graft for the production of the stent graft.
5. Physical manufacture of the stent graft with the aid of the optimized digital representation.

The proposed system possesses the following inventive characteristics:
The technical device described enables steps 1-5 to be performed at a workstation, with interfaces being present to allow data exchange with the subsystems. The system provides data export and data import functions so that individual steps can be performed on physically remote systems.
In step 4 improvements to the shape of the stent (bends, distortions, rotations, positioning of holes for outgoing vessels emerging from the aorta) can be made interactively on the screen, which improvements affect the digital representation of the stent graft and are taken into account during the manufacture of the stent.

Following the production or selection of the stent the physician positions the stent in the aortic aneurysm. The proposed system possesses the following inventive characteristics:
The system permits 3D volume data of the aortic aneurysm that was recorded before the intervention to be overlaid with 3D data (3D rotation angiography of the aorta) acquired during the intervention. This enables a possible progression of the disease between the recordings to be registered (the two recordings can be separated in time by days).
The difference between the two volume datasets can be represented, for example, by subtraction of one of the datasets from the other and visual presentation of the difference.
The system permits the image data recorded during the intervention (e.g. x-ray images, ultrasound images, 3D rotation angiography data) to be fused with volume data recorded prior to the intervention and/or the digital representation of the aortic aneurysm and/or the digital representation of the stent graft. In this way the current position of the (partially) expanded stent graft can be compared with the planned position of the stent graft, thus enabling an optimal positioning to be achieved.
The system permits the image data of the stent graft recorded during the intervention to be fused with the digital representation of the stent graft. This enables the actual position and expansion of the stent graft to be compared with the planned position and expansion.
Subsequently recorded diagnostic volume data can be fused with previously recorded volume data and/or the digital representation of the aortic aneurysm, with the digital representation of the stent graft and/or the image data recorded during the intervention.

The solution proposed here enables an optimal integration of diagnosis, therapy planning, therapeutic treatment, progress monitoring and documentation of the aortic aneurysm procedure.

Further advantages, features and details of the invention will emerge from the following description of an exemplary embodiment and with reference to the drawings, in which:

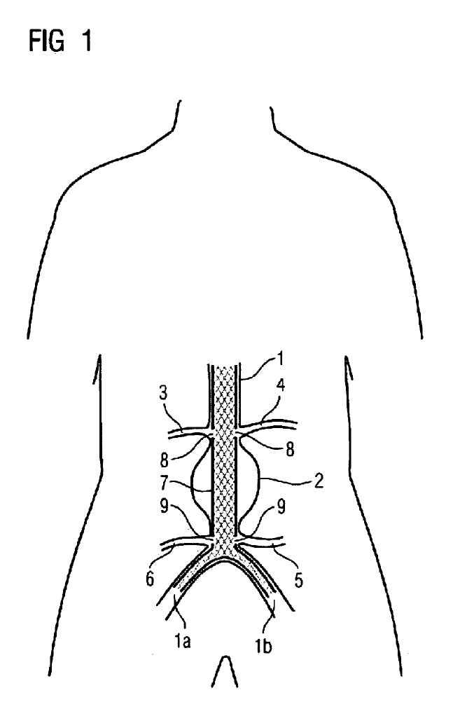

FIG. 1 shows a schematic representation of an aortic aneurysm with outgoing blood vessels and an implanted stent graft,

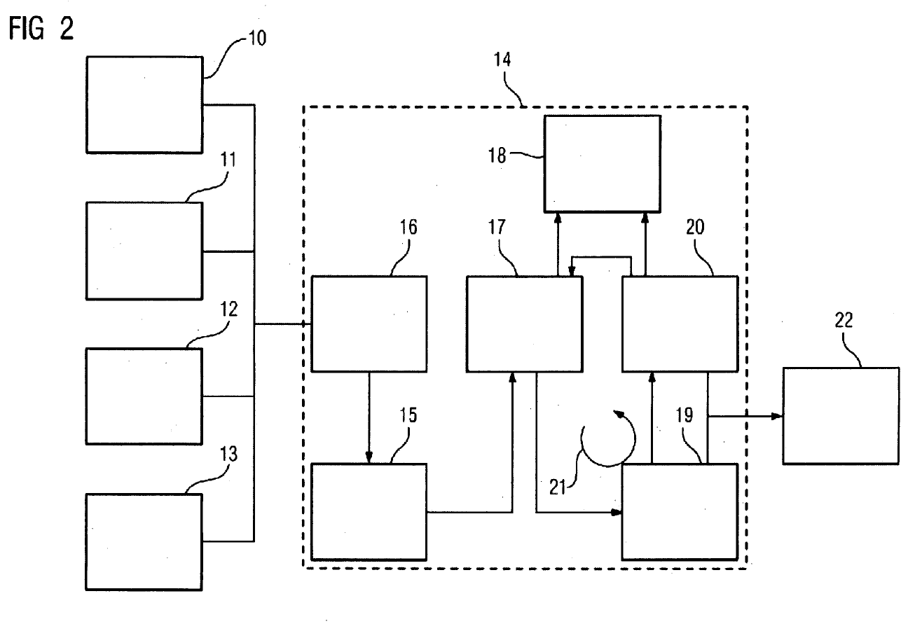

FIG. 2 shows a block diagram of the system according to the invention for treating an aortic aneurysm and for planning and manufacturing the stent graft required for the treatment; and

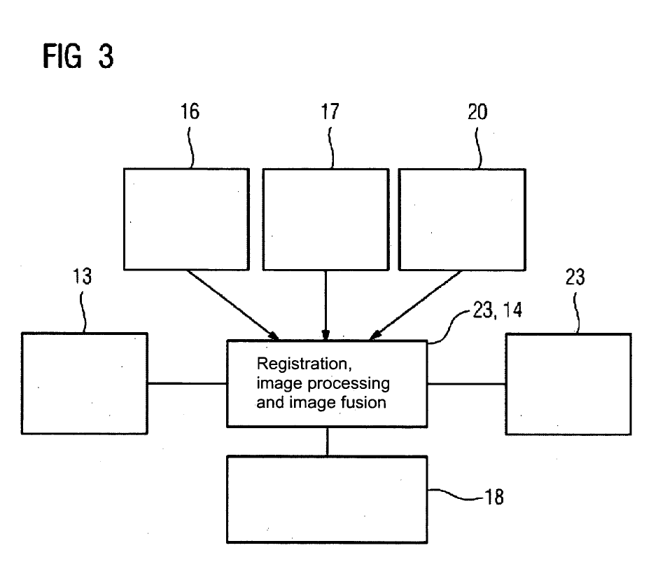

FIG. 3 shows a schematic representation of the system components during the intervention to implant the stent graft.

FIG. 1 shows a section from an aorta 1 in which a dilatation referred to as an aortic aneurysm 2 has formed, with a plurality of outgoing vessels 3-6 being provided in the area of said aortic aneurysm 2 and in addition a branching of the aorta behind the aortic aneurysm also being indicated. A stent graft 7 to be inserted for the purpose of treating said aortic aneurysm 2 must be provided with corresponding cutouts 8 and 9 in the area of the outgoing vessels 3-6 and in addition of course be adapted in terms of its dimensions both to the diameter of the aorta and to the diameters and the course of the branches.

In order to be able to plan and manufacture a made-to-measure stent graft, there is provided according to the invention a system as indicated schematically in FIG. 2.

In order to treat the aortic aneurysm use is made of a computer tomograph 10, a magnetic resonance tomography device 11, an ultrasound device 12 or an angiography device, for example a 2D or 3D rotation angiography device 13, with the preferably 3D volume data of an imaging device of said kind being supplied to a workstation 14, that is to say an integrated workplace. Using segmentation software 15, said workstation comprising memory devices builds a digital model 17 of the aortic aneurysm from the 3D volume data stored in the memory 16, which digital model 17 can be displayed on a display device 18.

A digital model 20 of a stent graft is created from the digital model of the aortic aneurysm by means of iterative customizing software 19, a repetition and iterative customizing being able to take place disposed through the loop 21. Following completion of the digital model of the stent graft the corresponding data can be supplied to an automatic or partially automated system 22 for producing vascular prostheses from digital representations in order to manufacture a made-to-measure stent graft corresponding to the digital model of the stent graft obtained on the basis of the digital model of the aortic aneurysm. An automatic or partially automatic system 22 for producing vascular prostheses can comprise, for example, a metal bending device, a CAD-controlled milling cutter or a plastic injection molding device or suchlike, a device of said kind being preferably arranged in the vicinity of the workstation 14, but where necessary also being networked with the workstation 14 at the premises of a manufacturer that, with the aid of the data obtained in the workstation, takes charge of the actual manufacture and is able to supply the stent graft as quickly as possible and closely linked in time with the diagnosis and planning of the stent graft, thereby ensuring that no further significant changes in the aortic aneurysm will have occurred in the meantime, in which event the stent graft would then possibly no longer fit precisely.

FIG. 3 shows in a schematic representation the integration of the systems during the intervention to implant the stent graft, with the intraoperative image data originating for example from an angiography device 13 and the physiological measurement data in the measurement unit 23 being able to comprise for example the respiration, the heartbeat or suchlike, the respiration and the heartbeat in particular being required for triggering in respect of the 3D volume data. At the same time a registration, image processing and image fusion of the individual elements of volume data and digital models takes place in the unit 23 14, as already discussed in detail in the foregoing, thus enabling the current position of the (partially) expanded stent graft to be compared with the planned position of the stent graft during the insertion and hence an optimal positioning to be achieved.

1.-7. (canceled)

8. A method of diagnosing and treating aortic aneurysms, comprising:
generating three-dimensional volume data of an area of an aorta affected by an aortic aneurysm for diagnosing the aortic aneurysm, by an imaging device;
developing a first digital model of the aortic aneurysm based on the three-dimensional volume data;
developing a second digital model of a stent graft based on the first digital model using an iterative customizing software, the stent graft configured to be inserted into the aorta in a folded state and to be expanded within the aorta;
manufacturing the stent graft based on the second digital model;
inserting the manufactured stent graft into the affected area of the aorta, the manufactured stent graft being in the folded state; and
expanding the stent graft within the affected area for treating the aortic aneurysm.

9. The method as claimed in claim 8, further comprising:
generating image data of the aorta during inserting the manufactured stent graft; and
merging the image data of the aorta with the first and/or second digital model.

10. The method as claimed in claim 9, wherein inserting the manufactured stent graft is supported by a medical imaging device chosen from the group consisting of a CT device, an MR device and an angiography device.

11. A device for diagnosing and treating aortic aneurysms, comprising:
an imaging device for generating three-dimensional volume data of an area of an aorta affected by an aortic aneurysm for diagnosing the aortic aneurysm;
an automatic or partially automated manufacturing station for manufacturing a stent graft configured to be inserted into the aorta in a folded state and to be expanded within the aorta;
a workstation connected to the imaging device and the manufacturing station, the workstation comprising:
a segmentation stage configured to develop a first digital model of the aortic aneurysm based on the three-dimensional volume data; and
an iteratively operating customizing stage for developing a second digital model of the stent graft based on the first digital model;
a display device for visualizing the three-dimensional volume data and/or the first and/or second digital model; and
a memory device for storing user data and control software, wherein the stent graft is manufactured by the manufacturing station based on the second digital model.

12. The device as claimed in claim 11, further comprising at least one medical imaging device chosen from the group consisting of a CT device, an MR device, an X-ray angiography system and an ultrasound system, the at least one medical imaging device connected to the workstation and configured to support inserting the manufactured stent graft into the affected area of the aorta by generating medical images of the aorta while inserting the manufactured stent graft into the affected area.

13. The device as claimed in claim 12, wherein the at least one medical imaging device is further configured to measure a blood flow through the aorta.

14. The device as claimed in claim 12, wherein the at least one medical imaging device is further configured to capture, edit, reconstruct, communicate and archive two-dimensional and/or three dimensional and/or four-dimensional image data.

15. The device as claimed in claim 11, wherein the imaging device is configured to merge medical image data including the three-dimensional volume data, the first and/or second digital model and physiological measurement data acquired from a patient undergoing aortic aneurysm treatment.

16. The device as claimed in claim 15, wherein the physiological measurement data are measurement data chosen from the group consisting of ECG data, blood pressure, blood flow, ventilation data acquired by an ventilator connected to the patient and anesthesia data acquired by an anesthesia system connected to the patient.

17. The device as claimed in claim 11, further comprising a correction module for ECG or breath gating to suppress artifacts.
